(19) United States
(12) Patent Application Publication
(10) Pub. No.: US 2008/0103461 A1
(43) Pub. Date: May 1, 2008

(54) Appliance for delivering a composition, the appliance having an outer fibrous layer and inner liquid-impermeable layer

(76) Inventors: Kroy D. Johnson, Menasha, WI (US); Kenneth B. Close, New London, WI (US)

Correspondence Address: KIMBERLY-CLARK WORLDWIDE, INC.;Catherine E. Wolf, 401 NORTH LAKE STREET, NEENAH, WI, 54956, US

(21) Appl. No.: 11/590,444

(22) Filed: Oct. 31, 2006

(51) Int. Cl. A61M 35/00

(52) U.S. Cl. 604/292; 604/293

(57) ABSTRACT

An appliance, such as a sleeve, patch, sock, or glove, that comprises a liquid-impermeable layer, an outer fibrous layer, and a composition or formulation associated with the interior or body-facing side of an appliance, effectively and comfortably treats the skin or tissue of a user. Humectants, materials of an occlusive nature, and numerous other ingredients may be included in the formulation. The outer fibrous layer helps provide the appliance with a cottony-soft feel and/or appearance.

### BACKGROUND

People rely on various kinds of formulations or compositions for health and/or hygiene benefits.

Generally, two categories of formulations are used when moisturizing and/or hydrating skin. Humectants are used to transport moisture from the environment (primarily water vapor in air) to skin. One example of a humectant is glycerin. Such materials tend to be hydrophilic (i.e., water loving), and are generally non-greasy, light in weight and/or viscosity, and quick to apply. Occlusive materials are used to trap moisture already present in the skin. These materials tend to create a water barrier over the skin, thereby inhibiting the transport of water from the skin to the external environment. An example of an occlusive material is petrolatum. Such materials tend to be heavier, effective over longer periods of time, and often contain oily and/or greasy ingredients.

Often people apply such formulations directly to skin using their hands. If the formulation includes water as an ingredient, water may evaporate, potentially decreasing the effectiveness of the formulation. Furthermore, after application of the formulation to the body, any excess formulation remaining on the hands must be removed.

In some instances, gloves, socks, sleeves, or other appliances have been used in conjunction with formulations. For example, a user either applies a formulation to his or her hand or foot, and then dons or applies a glove over the treated hand or a sock over the treated foot. Alternatively, a user slips on a glove or sock that has been pre-treated with a skin-care formulation. Unfortunately, such items have typically been made of a polymeric material (e.g., neoprene rubber) lacking a cloth-like appearance and feel. Often such items do not conform readily to the complex surfaces and contours of a foot or hand.

What is needed is an appliance that promotes health and/or hygiene by facilitating transport of a formulation or composition to skin or tissue, and which: has a liquid-impermeable layer that may be adapted to conform readily to the contours and surfaces of parts of the body to which the appliance is applied; and which has a fibrous outer layer to help promote a soft, cloth-like feel and appearance.

### SUMMARY

We have determined that an appliance, such as a sleeve, sock, or glove, that comprises liquid-impermeable layer, an outer fibrous layer, and a composition or formulation associated with the interior or body-facing side of an appliance, effectively and comfortably treats the skin or tissue of a user. Humectants, materials of an occlusive nature, and numerous other ingredients may be included in the formulation, examples of which are provided below in the Description section.

In one embodiment of the present invention, the liquid-impermeable layer comprises a film. The film is attached to an outer fibrous substrate. This outer substrate can impart a cloth-like appearance and feel to the appliance. In one version of the present invention, the outer fibrous substrate is attached to the film at discrete points or regions (as with, e.g., thermal point bonding). In another version of the present invention, the film is in a stretched condition when the outer fibrous substrate is bonded to it (e.g., at discrete points or regions), and then allowed to retract, thereby helping effect an increased rugosity or increase in the number of undulations associated with the outer fibrous layer.

In another version of the present invention, the aforementioned film comprises an elastomeric polymer, thereby imparting elasticity to the appliance as a whole. As noted above, a film employing an elastomeric material, or materials, may be attached to the outer fibrous layer either uniformly, or at selected locations, including discrete points or regions (e.g., as result from thermal point bonding). Furthermore, a film employing elastomeric material(s) may be stretched prior to bonding or attachment to the outer fibrous layer, and then allowed to retract.

These and other versions of the invention are described more fully below.

### DRAWINGS

FIG. 1 representatively illustrates one version of a substrate of the present invention.

FIG. 2 representatively illustrates one version of a substrate cut so that the substrate's perimeter defines the shape of a hand.

FIG. 2A representatively illustrates one version of an appliance of the present invention.

FIG. 3 representatively illustrates one version of a substrate cut so as to form a foot appliance of the present invention.

FIG. 3A representatively illustrates one version of an appliance of the present invention.

### DEFINITIONS

Within the context of this specification, each term or phrase below includes the following meaning or meanings:

“Attach” and its derivatives refer to the joining, adhering, connecting, bonding, sewing together, depositing on, associating with, or the like, of two elements. Two elements will be considered to be attached together when they are integral with one another or attached directly to one another or indirectly to one another, such as when each is directly attached to intermediate elements. “Attach” and its derivatives include permanent, releasable, or refastenable attachment. In addition, the attachment can be completed either during the manufacturing process or by the end user.

“Bond” and its derivatives refer to the joining, adhering, connecting, attaching, sewing together, or the like, of two elements. Two elements will be considered to be bonded together when they are bonded directly to one another or indirectly to one another, such as when each is directly bonded to intermediate elements. “Bond” and its derivatives include permanent, releasable, or refastenable bonding.

“Coform” refers to a blend of meltblown fibers and absorbent fibers such as cellulosic fibers that can be formed by air forming a meltblown polymer material while simultaneously blowing air-suspended fibers into the stream of meltblown fibers. The coform material may also include other materials, such as superabsorbent materials. The meltblown fibers and absorbent fibers are collected on a forming surface, such as provided by a foraminous belt. The forming surface may include a gas-pervious material that has been placed onto the forming surface.

“Composition,” “formulation,” or their derivatives, when used in the context of a material applied or deposited on the interior surface of an appliance of the present invention, refers to the various materials that help improve the health and/or hygiene of a user of the appliance, primarily by helping improve the health and/or hygiene of skin or tissue to which the material is transferred from the interior surface of the appliance.

“Connect” and its derivatives refer to the joining, adhering, bonding, attaching, sewing together, or the like, of two elements. Two elements will be considered to be connected together when they are connected directly to one another or indirectly to one another, such as when each is directly connected to intermediate elements. “Connect” and its derivatives include permanent, releasable, or refastenable connection. In addition, the connecting can be completed either during the manufacturing process or by the end user.

“Disposable” refers to articles which are designed to be discarded after a limited use rather than being laundered or otherwise restored for reuse.

The terms “disposed on,” “disposed along,” “disposed with,” or “disposed toward” and variations thereof are intended to mean that one element can be integral with another element, or that one element can be a separate structure bonded to or placed with or placed near another element.

“Fiber” refers to a continuous or discontinuous member having a high ratio of length to diameter or width. Thus, a fiber may be a filament, a thread, a strand, a yarn, or any other member or combination of these members.

“Hydrophilic” describes materials or surfaces which are wetted by aqueous liquids in contact with the material or surface. The degree of wetting of the material or surface can, in turn, be described in terms of the contact angles and the surface tensions of the liquids and materials (or surfaces) involved.

“Layer” when used in the singular can have the dual meaning of a single element or a plurality of elements.

“Liquid impermeable,” when used in describing a layer or multi-layer laminate means that liquid, such as water, will not pass to any appreciable extent through the layer or laminate, under ordinary use conditions, in a direction generally perpendicular to the plane of the layer or laminate at the point of liquid contact.

“Liquid permeable” refers to any material that is not liquid impermeable.

“Meltblown” refers to fibers formed by extruding a molten thermoplastic material through a plurality of fine, usually circular, die capillaries as molten threads or filaments into converging high velocity gas (e.g., air) streams, generally heated, which attenuate the filaments of molten thermoplastic material to reduce their diameters. Thereafter, the meltblown fibers are carried by the high velocity gas stream and are deposited on a collecting surface to form a web of randomly dispersed meltblown fibers. Such a process is disclosed, for example, in U.S. Pat. No. 3,849,241 to Butin et al. Meltblowing processes can be used to make fibers of various dimensions, including macrofibers (with average diameters from about 40 to about 100 microns), textile-type fibers (with average diameters between about 10 and 40 microns), and microfibers (with average diameters less than about 10 microns). Meltblowing processes are particularly suited to making microfibers, including ultra-fine microfibers (with an average diameter of about 3 microns or less). A description of an exemplary process of making ultra-fine microfibers may be found in, for example, U.S. Pat. No. 5,213,881 to Timmons, et al. Meltblown fibers may be continuous or discontinuous and are generally self bonding when deposited onto a collecting surface.

“Member” when used in the singular can have the dual meaning of a single element or a plurality of elements.

“Nonwoven” and “nonwoven web” refer to materials and webs of material that are formed without the aid of a textile weaving or knitting process. For example, nonwoven materials, fabrics or webs have been formed from many processes such as, for example, meltblowing processes, spunbonding processes, air laying processes, and bonded carded web processes.

“Spunbonded fibers” refers to small diameter fibers which are formed by extruding molten thermoplastic material as filaments from a plurality of fine, usually circular capillaries of a spinneret with the diameter of the extruded filaments then being rapidly reduced to fibers as by, for example, in U.S. Pat. No. 4,340,563 to Appel et al., and U.S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat. No. 3,502,763 to Hartman, and U.S. Pat. No. 3,542,615 to Dobo et al., the contents of which are incorporated herein by reference in their entirety. Spunbond fibers are generally continuous and have diameters generally greater than about 7 microns, more particularly, between about 10 and about 20 microns.

“Stretch bonded laminate” refers to a composite material having at least two layers in which one layer is a gatherable layer and the other layer is an elastic layer. The layers are joined together when the elastic layer is extended from its original condition so that upon relaxing the layers, the gatherable layer is gathered. Such a multilayer composite elastic material may be stretched to the extent that the nonelastic material gathered between the bond locations allows the elastic material to elongate. One type of stretch bonded laminate is disclosed, for example, by U.S. Pat. No. 4,720,415 to VanderWielen et al., the content of which is incorporated herein by reference in its entirety. Other composite elastic materials are disclosed in U.S. Pat. No. 4,789,699 to Kieffer et al., U.S. Pat. No. 4,781,966 to Taylor and U.S. Pat. Nos. 4,657,802 and 4,652,487 to Morman and U.S. Pat. No. 4,655,760 to Morman et al., the contents of which are incorporated herein by reference in their entirety.

“Necking” or “neck stretching” interchangeably refer to a method of elongating a nonwoven fabric, generally in the machine direction, to reduce its width (cross-machine direction) in a controlled manner to a desired amount. The controlled stretching may take place under cool, room temperature or greater temperatures and is limited to an increase in overall dimension in the direction being stretched up to the elongation required to break the fabric, which in most cases is about 1.2 to 1.6 times. When relaxed, the web retracts toward, but does not return to, its original dimensions. Such a process is disclosed, for example, in U.S. Pat. No. 4,443,513 to Meitner and Notheis, U.S. Pat. Nos. 4,965,122, 4,981,747 and 5,114,781 to Morman and U.S. Pat. No. 5,244,482 to Hassenboehier Jr. et al., the contents of which are incorporated herein by reference in their entirety.

“Necked material” refers to any material which has undergone a necking or neck stretching process.

“Reversibly necked material” refers to a material that possesses stretch and recovery characteristics formed by necking a material, then heating the necked material, and cooling the material. Such a process is disclosed in U.S. Pat. No. 4,965,122 to Morman, commonly assigned to the assignee of the present invention, and incorporated by reference herein in its entirety. As used herein, the term “neck bonded laminate” refers to a composite material having at least two layers in which one layer is a necked, non-elastic layer and the other layer is an elastic layer. The layers are joined together when the non-elastic layer is in an extended (necked) condition. Examples of neck-bonded laminates are such as those described in U.S. Pat. Nos. 5,226,992, 4,981,747, 4,965,122 and 5,336,545 to Morman, the contents of which are incorporated herein by reference in their entirety.

“Stitchbonded” refers to a process in which materials (fibers, webs, films, etc.) are joined by stitches sewn or knitted through the materials. Examples of such processes are illustrated in U.S. Pat. No. 4,891,957 to Strack et al. and U.S. Pat. No. 4,631,933 to Carey, Jr., the contents of which are incorporated herein by reference in their entirety.

“Ultrasonic bonding” refers to a process in which materials (fibers, webs, films, etc.) are joined by passing the materials between a sonic horn and anvil roll. An example of such a process is illustrated in U.S. Pat. No. 4,374,888 to Bornslaeger, the content of which is incorporated herein by reference in its entirety.

“Thermal point bonding” involves passing materials (fibers, webs, films, etc.) to be bonded between a heated calender roll and an anvil roll. The calender roll is usually, though not always, patterned in some way so that the entire fabric is not bonded across its entire surface, and the anvil roll is usually flat. As a result, various patterns for calender rolls have been developed for functional as well as aesthetic reasons. Typically, the percent bonding area varies from around 10 percent to around 30 percent of the area of the fabric laminate. As is well known in the art, thermal point bonding holds the laminate layers together and imparts integrity to each individual layer by bonding filaments and/or fibers within each layer.

“Elastic” refers to any material, including a film, fiber, nonwoven web, or combination thereof, which upon application of a biasing force in at least one direction, is stretchable to a stretched, biased length which is at least about 110 percent, suitably at least about 130 percent, and particularly at least about 150 percent, its relaxed, unstretched length, and which will recover at least 15 percent of its elongation upon release of the stretching, biasing force. In the present application, a material need only possess these properties in at least one direction to be defined as elastic.

“Extensible and retractable” refers to the ability of a material to extend upon stretch and retract upon release. Extensible and retractable materials are those which, upon application of a biasing force, are stretchable to a stretched, biased length and which will recover a portion, suitably at least about 15 percent, of their elongation upon release of the stretching, biasing force.

As used herein, the terms “elastomer” or “elastomeric” refer to polymeric materials that have properties of stretchability and recovery.

“Stretch” refers to the ability of a material to extend upon application of a biasing force. Percent stretch is the difference between the initial dimension of a material and that same dimension after the material has been stretched or extended following the application of a biasing force. Percent stretch may be expressed as [(stretched length−initial sample length)/initial sample length]×100. For example, if a material having an initial length of one (1) inch is stretched 0.50 inch, that is, to an extended length of 1.50 inches, the material can be said to have a stretch of 50 percent.

“Recover” or “recovery” refers to a contraction of a stretched material upon termination of a biasing force following stretching of the material by application of the biasing force. For example, if a material having a relaxed, unbiased length of one (1) inch is elongated 50 percent by stretching to a length of one and one half (1.5) inches the material would have a stretched length that is 150 percent of its relaxed length. If this exemplary stretched material contracted, that is recovered to a length of one and one tenth (1.1) inches after release of the biasing and stretching force, the material would have recovered 80 percent (0.4 inch) of its elongation.

“Water impermeable,” when used in describing a layer or multi-layer laminate means that water or water vapor will not pass to any appreciable extent through the layer or laminate, under ordinary use conditions, in a direction generally perpendicular to the plane of the layer or laminate at the point of liquid contact.

“Water permeable” refers to any material that is not water impermeable.

These terms may be defined with additional language in the remaining portions of the specification.

### DESCRIPTION

Various problems associated with skin or tissue may diminish the health and/or hygiene of a person or animal. For example, dryness of the skin of the hands, feet, extremities, joints, or other parts of a body is a common problem, especially when skin is exposed to cold and/or dry conditions. This may be especially true for older individuals. Various formulations designed to moisturize skin are often used to address this problem. Some formulations require a substantial amount of time to deliver the anticipated benefit. For many currently available formulations, any resulting skin benefit may last a relatively short period of time after the formulation has been applied. The present invention characterizes an appliance for delivering a formulation or composition to tissue or skin.

One example of such a composition is a skin-care formulation for moisturizing skin. Such formulations typically work through at least one of two mechanisms: occlusivity or humectancy. Skin-care formulations relying on occlusivity form a relatively water-vapor-impermeable film on and/or in a skin surface. This occlusive film results in the accumulation of water underneath the film as the skin undergoes the natural process of trans-epidermal water loss. One advantage of the occlusivity approach includes the ability to provide a skin-moisturization benefit for an extended period of time. Occlusive ingredients are typically hydrophobic in nature and are generally not easily washable, which contribute to their ability to provide long-term moisturization of skin. Formulations with ingredients having occlusive properties (such as peterolatum, waxes, vegetable oils, mineral oil, etc.) are perceived by some as having undesirable aesthetic and/or feel attributes. Occlusive formulations may also lack the ability to provide quick moisturization as such formulations depend on the relatively slow process of water accumulation due to trans-epidermal water loss to deliver the moisturizing benefit.

Humectant formulations have the ability to attract water vapor (moisture) from the atmosphere and bring it to the skin surface, which results in increased skin hydration and alleviation of dryness. This process of attracting moisture is frequently referred to as “hygroscopicity”. Humectant formulations have the advantage of delivering a moisturization benefit and dryness relief within a short period of time. Humectant formulations are typically hydrophilic (as noted above, “water loving”) in nature and generally contain a significant amount of water. Such formulations are generally perceived as having a light, pleasant feel (i.e., light in weight and/or viscosity) on the skin and typically are aesthetically preferred by the user (relative to skin-care compositions that function by occlusivity). Examples of humectant ingredients include glycerin, urea, sodium lactate, polysaccharides, and the like. Unlike occlusive formulations, humectant formulations generally lack the ability to provide moisturization over an extended period of time.

In one aspect of the present invention, an aqueous formulation comprising a humectant is applied or associated with the interior or body-facing side of an appliance such as a glove, sock, or sleeve. The appliance comprises a liquid-impermeable layer, such as a film, that prevents, reduces, or minimizes transmission of water through the appliance to the external environment. The liquid-impermeable layer can comprise elastomeric polymers, and may be formed in such a way, that the liquid-impermeable film is also elastomeric. Such polymers include, for example, KRATON styrenic block copolymers available from businesses such as Kraton, Kurary, and Dynasol; DEXCO olefinic polymers available from businesses such as Dow Chemical and ExxonMobil; and other such polymers.

The film is attached to an outer fibrous layer. Without being bound to a particular theory, we believe an appliance having this configuration and comprising a humectant-type formulation can realize the benefits of both humectancy and occlusivity without their respective disadvantages. A humectant-type formulation on the inside of the appliance provides the aesthetically pleasing feel to the appliance user and delivers the initial quick moisturization benefit to the skin. The occlusive nature of the liquid-impermeable film in the product contributes to a longer-term moisturization effect. Furthermore, the outer fibrous layer can give the appliance a cloth-like feel and appearance. As discussed elsewhere, undulations or texture may be effected, in whole or in part, by attaching the outer fibrous layer to the liquid-impermeable layer at discrete points or locations while the liquid-impermeable layer is in a stretched condition. When the resulting laminate is allowed to contract, the undulations or texture are associated with the outer fibrous layer. Furthermore, to the extent the liquid-impermeable film employs elastomeric materials, the resulting appliance is able to stretch and more readily conform to the skin surface. And if the outer fibrous layer is bonded at discrete locations to the elastomeric liquid-impermeable layer while in a stretched condition, and then allowed to retract, thereby partially gathering the outer fibrous layer, then the appliance is especially well suited to being adapted to stretch and conform to complex skin surfaces.

### Representative Substrates for Constructing an Appliance of the Present Invention

A substrate used to make an appliance of the present invention will generally have at least two members: a liquid- or water-impermeable layer (such as an elastomeric film); and an outer fibrous layer.

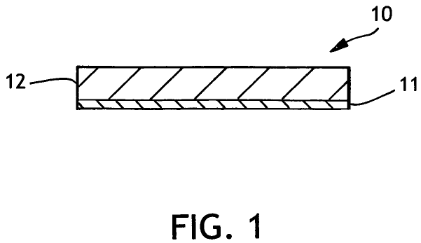

An example of such a substrate 10 is depicted in FIG. 1, which representatively illustrates a liquid-impermeable layer 12 attached to a fibrous layer 14. In the example depicted in FIG. 1, the liquid-impermeable layer 12 is a film. A suitable class of film materials includes a thermoplastic elastomeric polyolefin polymer. These (and other) components can be mixed together, heated, and then extruded into a mono-layer or multi-layer film using any one of a variety of film-producing processes known to those of ordinary skill in the film processing art. Such film-making processes include, for example, cast embossed, chill and flat cast, and blown film processes. Typically the liquid-impermeable layer 12 will be attached to the fibrous layer 14 using an adhesive, thermal bonding, ultrasonic bonding, or the like.

Other additives and ingredients may be added to the liquid-impermeable layer 12 provided they do not significantly interfere with the ability of the liquid-impermeable layer to function in accordance with the teachings of the present invention. Such additives and ingredients can include, for example, antioxidants, stabilizers, and pigments.

In addition to the polyolefin polymer in this representative example, a liquid-impermeable layer 12 can also include a filler. As used herein, a “filler” is meant to include particulates and other forms of materials that can be added to the film polymer extrusion blend and that will not chemically interfere with the extruded film but which are able to be uniformly dispersed throughout the film. Generally, the fillers will be in particulate form and may have a spherical or non-spherical shape with average particle sizes in the range of about 0.1 to about 7 microns. Both organic and inorganic fillers are contemplated to be within the scope of the present invention provided that they do not interfere with the film formation process, or the ability of the film layer to function in accordance with the teachings of the present invention. Examples of suitable fillers include calcium carbonate (CaCO₃), various kinds of clay, silica (SiO₂), alumina, barium carbonate, sodium carbonate, magnesium carbonate, talc, barium sulfate, magnesium sulfate, aluminum sulfate, titanium dioxide (TiO₂), zeolites, cellulose-type powders, kaolin, mica, carbon, calcium oxide, magnesium oxide, aluminum hydroxide, pulp powder, wood powder, cellulose derivatives, chitin and chitin derivatives. A suitable coating, such as, for example, stearic acid, may also be applied to the filler particles.

As mentioned herein, liquid-impermeable layer 12 may be formed using any one of the conventional processes known to those familiar with film formation, if the liquid-impermeable layer is composed of a film. If so, a polyolefin or other polymer and any optional ingredients (e.g., filler) are mixed in and then heated and extruded into a film.

As noted above, the liquid-impermeable layer serves to impede or reduce the amount of water and/or water vapor that will migrate or diffuse through the layer. In this way, as discussed elsewhere, the liquid-impermeable layer serves to act as an occlusive material, i.e., the liquid-impermeable layer promotes the retention of, for example, water in a formulation or composition. Typically a film will be liquid-impermeable, unless it is perforated; or made breathable (i.e., made to allow the passage of water vapor) by, e.g., the addition of a sufficient amount of filler or particulate to the material inputs to film formation; or is otherwise altered so that liquid is able to readily pass through it.

If a nonwoven material is used to make the outer fibrous layers, then commercially available thermoplastic polymeric materials can be advantageously employed in making the fibers or filaments from which the fibrous layer 14 is formed. As used herein, the term “polymer” shall include, but is not limited to, homopolymer, copolymers, such as, for example, block, graft, random and alternating copolymers, terpolymers, etc., and blends and modifications thereof. Moreover, unless otherwise specifically limited, the term “polymer” shall include all possible geometric configurations of the material, including, without limitation, isotactic, syndiotactic, random and atactic symmetries. As used herein, the terms “thermoplastic polymer” or “thermoplastic polymeric material” refer to a long-chain polymer that softens when exposed to heat and returns to the solid state when cooled to ambient temperature. Exemplary thermoplastic materials include, without limitation, polyvinyl chlorides, polyesters, polyamides, polyfluorocarbons, polyolefins, polyurethanes, polystyrenes, polyvinyl alcohols, caprolactams, and copolymers of the foregoing.

Nonwoven webs that can be employed as fibrous layer 14 of the present invention can be formed by a variety of known forming processes, including spunbonding, airlaying, meltblowing, or bonded carded web formation processes. Spunbond nonwoven webs are made from melt-spun filaments. As used herein, the term “meltspun filaments” refers to small diameter fibers and/or filaments which are formed by extruding a molten thermoplastic material as filaments from a plurality of fine, usually circular, capillaries of a spinneret with the diameter of the extruded filaments then being rapidly reduced, for example, by non-eductive or eductive fluid drawing or other well known spunbonding mechanisms. Lastly, the melt-spun filaments are deposited in a substantially random manner onto a moving carrier belt or the like to form a web of substantially continuous and randomly arranged, melt-spun filaments. Spunbond filaments generally are not tacky when they are deposited onto the collecting surface. The production of spunbond nonwoven webs is described in U.S. Pat. No. 4,340,563 to Appel et al., U.S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat. No. 3,502,538 to Peterson, and U.S. Pat. No. 3,542,615 to Dobo et al., all of which are incorporated herein by reference. The melt-spun filaments formed by the spunbond process are generally continuous and have average diameters larger than 7 microns based upon at least 5 measurements, and more particularly, between about 10 and 100 microns. Another frequently used expression of fiber or filament diameter is denier, which is defined as grams per 9000 meters of a fiber or filament.

Spunbond webs generally are stabilized or consolidated (pre-bonded) in some manner immediately as they are produced in order to give the web sufficient integrity and strength to withstand the rigors of further processing into a finished product. This pre-bonding step may be accomplished through the use of an adhesive applied to the filaments as a liquid or powder which may be heat activated, or more commonly, by compaction rolls. As used herein, the term “compaction rolls” means a set of rollers above and below the nonwoven web used to compact the web as a way of treating a just produced, melt-spun filament, particularly spunbond, web, in order to give the web sufficient integrity for further processing, but not the relatively strong bonding of later applied, secondary bonding processes, such as through-air bonding, thermal bonding, ultrasonic bonding and the like. Compaction rolls slightly squeeze the web in order to increase its self-adherence and thereby its integrity.

An exemplary secondary bonding process utilizes a patterned roller arrangement for thermally bonding the spunbond web. The roller arrangement typically includes a patterned bonding roll and a smooth anvil roll which together define a thermal patterning bonding nip. Alternatively, the anvil roll may also bear a bonding pattern on its outer surface. The pattern roll is heated to a suitable bonding temperature by conventional heating means and is rotated by conventional drive means, so that when the spunbond web passes through the nip, a series of thermal pattern bonds is formed. Nip pressure within the nip should be sufficient to achieve the desired degree of bonding of the web, given the line speed, bonding temperature and materials forming the web. Percent bond areas within the range of from about 10 percent to about 20 percent are typical for such spunbond webs.

The liquid-impermeable layer 12 can be formed of any film to yield a substrate 10 having the performance characteristics and features described herein. A suitable class of film materials includes a thermoplastic elastomeric polyolefin polymer. These (and other) components can be mixed together, heated and then extruded into a mono-layer or multi-layer film using any one of a variety of film- producing processes known to those of ordinary skill in the film processing art. Such film-making processes include, for example, cast embossed, chill and flat cast, and blown film processes. The liquid-impermeable layer 12 will typically be attached to the fibrous layer 14 by thermally bonding the layers together at discrete points (see, e.g., above discussion as well as U.S. Pat. No. 6,037,281, entitled “Cloth-Like, Liquid-Impervious, Breathable Composite Barrier Fabric,” to Mathis, et al.). As noted above, the fibrous layer may be bonded or attached to the liquid-impermeable layer at discrete locations while the liquid-impermeable layer is in a stretched condition, thereby producing or increasing undulations when the resulting laminate is in a relaxed condition. Other known means for bonding and laminating the liquid-impermeable layer 12 to a fibrous layer may be used, provided the resulting substrate 10 has the required properties described herein. For example, the layers may be adhesively bonded to one another. Additional examples of exemplary processes for making an appliance of the present invention (i.e., a film associated with a fibrous layer but which, in accordance with the teaching of the present application, would orient the substrate such that the fibrous layer is oriented away from the skin or tissue of a user; and the film would be oriented toward the skin or tissue of a user, with a formulation or composition associated with the film such a “shielding layer,” as that term is used in the referenced application that follows, would be interposed between the formulation and any elastic/liquid-impermeable layer) are described in a co-pending U.S. patent application (patent application serial number not yet assigned), filed on the same day as the present application, 31 Oct. 2006, and corresponding to internal docket number 64343490US02. This application is entitled “Method for Making An Appliance for Delivering a Composition, The Appliance Having an Elastic Layer and a Shielding Layer,” to Kenneth Close, Jonathan Arendt, and Gary Anderson, and is hereby incorporated by reference in its entirety in a manner consistent herewith.

A suitable class of film materials includes a thermoplastic elastomeric polyolefin polymer, including, for example, polypropylene. Suitable propylene polymers are commercially available under the designations VISTAMAXX™ from ExxonMobil Chemical Co. of Houston, Tex.; FINA™ (e.g., 8573) from Atofina Chemicals of Feluy, Belgium; TAFMER™ available from Mitsui Petrochemical Industries; and VERSIFY™ available from Dow Chemical Co. of Midland, Mich. Other examples of suitable propylene polymers are described in U.S. Pat. No. 7,105,609 to Datta, et al.; U.S. Pat. No. 6,500,563 to Datta, et al.; U.S. Pat. No. 5,539,056 to Yang, et al.; and U.S. Pat. No. 5,596,052 to Resconi, et al., which are incorporated herein in their entirety by reference thereto for all purposes. Other examples of polymers that may be employed in liquid-impermeable layer are described in a co-pending U.S. provisional patent application, filed on 27 Sep. 2006, and corresponding to internal docket number 64048978US01. This application is entitled “Elastic Composite Having Barrier Properties,” to Laura Keck, et al., and is hereby incorporated by reference in its entirety in a manner consistent herewith.

Of course, other thermoplastic polymers may also be used to form the elastic film so long as they do not adversely affect the elastic properties of the film (or as discussed elsewhere, e.g., strands, or composites). For example, the elastic film may contain other polyolefins, elastomeric polyesters, polyurethanes, polyamides, block copolymers, and so forth. For example, polyethylene may be employed in some embodiments of the present invention. The density of the polyethylene may vary depending on the type of polymer employed, but generally ranges from 0.85 to 0.96 grams per cubic centimeter (“g/cm³”). Polyethylene “plastomers”, for instance, may have a density in the range of from 0.85 to 0.91 g/cm³. Likewise, “linear low density polyethylene” (“LLDPE”) may have a density in the range of from 0.91 to 0.940 g/cm³; “low density polyethylene” (“LDPE”) may have a density in the range of from 0.910 to 0.940 g/cm³; and “high density polyethylene” (“HDPE”) may have density in the range of from 0.940 to 0.960 g/cm³.

Besides polymers, the elastic film of the present invention may also contain other additives as is known in the art, such as melt stabilizers, processing stabilizers, heat stabilizers, light stabilizers, antioxidants, heat aging stabilizers, whitening agents, antiblocking agents, bonding agents, tackifiers, viscosity modifiers, etc. Suitable viscosity modifiers may include, for instance, polyethylene wax (e.g., EPOLENE™ C-10 from Eastman Chemical). Phosphite stabilizers (e.g., IRGAFOS available from Ciba Specialty Chemicals of Terrytown, N.Y. and DOVERPHOS available from Dover Chemical Corp. of Dover, Ohio) are exemplary melt stabilizers. In addition, hindered amine stabilizers (e.g., CHIMASSORB available from Ciba Specialty Chemicals) are exemplary heat and light stabilizers. Further, hindered phenols are commonly used as an antioxidant in the production of films. Some suitable hindered phenols include those available from Ciba Specialty Chemicals of under the trade name “Irganox®”, such as Irganox® 1076, 1010, or E 201. Moreover, bonding agents may also be added to the film to facilitate bonding of the film to additional materials (e.g., nonwoven web). When employed, such additives (e.g., tackifier, antioxidant, stabilizer, etc.) may each be present in an amount from about 0.001 wt. % to about 25 wt. %, in some embodiments, from about 0.005 wt. % to about 20 wt. %, and in some embodiments, from 0.01 wt. % to about 15 wt. % of the film.

The preceding paragraphs describe some examples of a substrate that may be used in an appliance of the present invention. For additional examples, see, e.g., U.S. Pat. No. 6,037,281, entitled “Cloth-Like, Liquid-Impervious, Breathable Composite Barrier Fabric,” to Mathis, et al.; U.S. Pat. No. 4,663,220 issued May 5, 1987 to Wisneski et al.; U.S. Pat. No. 5,226,992 issued Jul. 13, 1993 to Morman; European Patent Application No. EP 0 217 032 published on Apr. 8, 1987 in the name of Taylor et al.; and PCT Application WO 01/88245 in the name of Welch et al.; all of which are incorporated herein by reference in a manner consistent herewith.

### Representative Appliance Configurations

One or more substrates, such as those described above, may be configured into the form of a glove, mitten, sock, sleeve, patch, or other article designed to be fitted to a part of the body. Generally the appliance will be made by cutting a substrate into appropriate pieces such that the pieces, when attached to one another, form an appliance having an interior volume into which a portion of a body may be inserted. But, as noted above, the appliance may be configured in the form of a patch. Typically a formulation will be associated with the appliance during manufacture so that the appliance is ready to use. In some versions of the present invention, however, a formulation is not pre-applied to the appliance, allowing a user to choose and apply a formulation or composition to his or her skin, and then don or affix an appliance of the present invention to the skin or tissue to which the formulation or composition was applied.

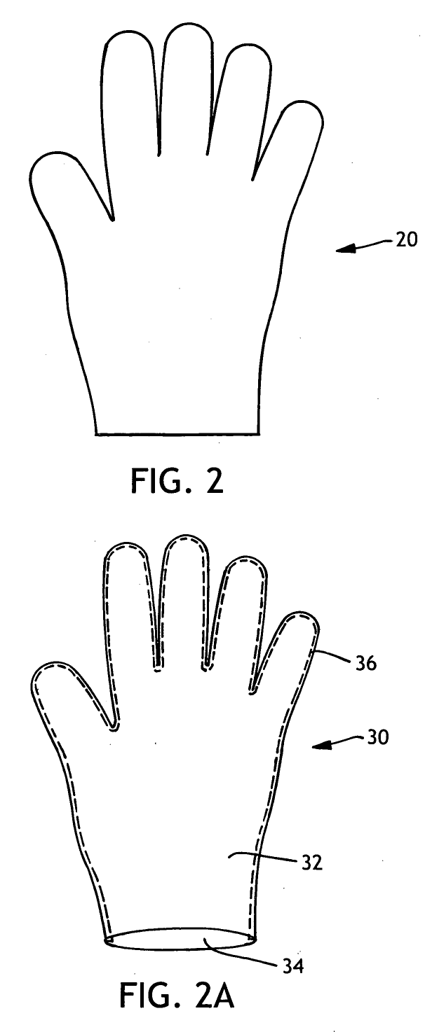

FIG. 2 representatively depicts a substrate 20 cut so that the piece (or substrate) defines a perimeter in the shape of a human hand. FIG. 2A representatively depicts an appliance 30 comprising a first piece (or substrate) 32 attached to a second piece (or substrate) 34 at a location proximate to the perimeters of these two substrates. In this representative illustration, the two substrates are attached to one another mechanically by sewing the pieces together at a location proximate to the perimeters of the two substrates. The resulting appliance was then inverted so that the seam 36 formed by sewing the substrates together is on the interior of the appliance. Of course the finished appliance need not be inverted; the seam can remain on the exterior of the appliance. Note, too, that the individual pieces need not be joined in a way that produces a seam. The edges of the individual pieces may be butted together, and then, for example, joined and/or welded together using a solvent. Alternatively, the individual pieces may be butted together, and another material, such as an adhesive or an adhesive tape, used to join the pieces together. Or the pieces may be thermally bonded or ultrasonically bonded. Furthermore, any glove-like appliance may be formed such that the appliance resembles a bicycle glove, or some portion thereof (i.e., one or more end portions of the individual thumb-like and/or finger-like projections of the glove-like appliance are absent, so that a person may more easily manipulate objects while wearing the appliance because some portion of one or more fingers and/or the thumb is exposed [and at the same time treat skin, for example, at joints, the back of the hand, the palm, or some combination thereof]). Alternatively, a sock may be formed such that a portion proximate to the heel, the toe(s), or some other portion of a user's foot is exposed.

Individual pieces (or substrates) may be cut into a variety of shapes and sizes. Rather than the glove depicted in FIGS. 2 and 2A, the pieces may be cut so that the resulting appliance is in the shape of a tube, sleeve, mitten, sock, or the like. Any shape is possible, so long as the resulting appliance defines an interior volume (for those versions of the invention in which the appliance defines an interior volume) into which a user may insert a portion of his or her body (e.g., a finger, toe, hand, foot, wrist, forearm, etc.) such that a composition applied to, or associated with, the interior surface of the appliance may be transferred to skin or tissue in contact with the interior surface of the appliance. As noted elsewhere, however, in some versions of the invention the appliance is a patch that is applied or affixed to skin (e.g., a patch comprising a body adhesive proximate to the perimeter of the patch, thereby allowing the patch to be releasably affixed to the skin). Furthermore, as stated elsewhere, in some versions of the invention the formulation or composition is applied separately to the skin, followed by a user employing an appliance of the present invention.

The individual substrates or pieces need not be sewn together. The individual pieces or substrates may also be joined ultrasonically, thermally, adhesively, cohesively, using tape, by fusing the materials together (e.g., by using an appropriate solvent), by welding the materials together, or by other approaches. So long as the individual pieces or substrates remain attached or connected during normal use of the appliance, and attachment or connection is such that any composition or formulation on the interior surface of the appliance is contained within the appliance (i.e., there is minimal or no leakage of the formulation or composition), any connection or attachment may be used.

Alternatively, a substrate could be prepared in the form of a rectangle, oval, or other shape (e.g., as for a patch). An adhesive capable of adhering to skin could then be applied to all or part of the perimeter of the shape such that the appliance could be releasably adhered to skin. Any composition to be transferred to skin could then be coated or deposited on the surface of the appliance that will contact skin or tissue.

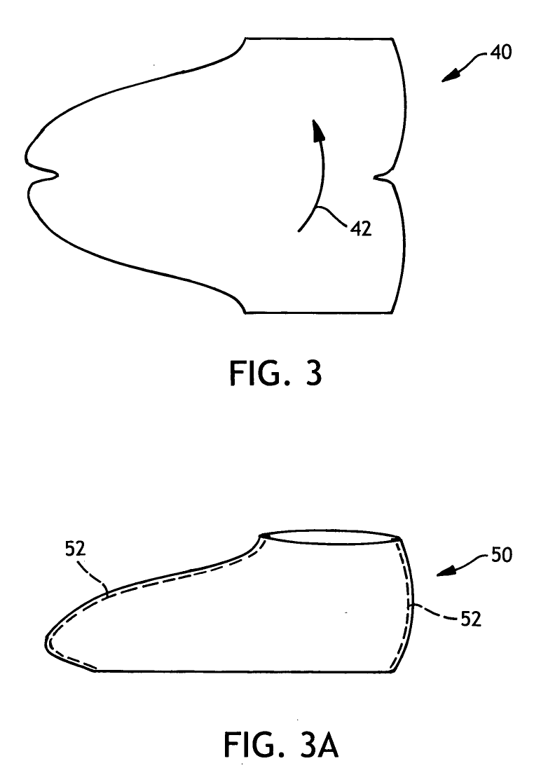

Note, too, that an appliance defining some interior volume may be formed from a single piece of substrate. In one exemplary embodiment, FIG. 3 representatively illustrates a substrate 40 that has been cut in a way that a foot-shaped appliance may be formed by folding the substrate back on itself (as shown by arrow 42; the bottom half of the shape is folded upward, and on top of, the top half of the shape). FIG. 3A representatively illustrates such a foot-shape appliance 50 and the resulting seams 52 formed when the substrate 40 (from FIG. 3) is folded back, and attached to, itself. In this representative embodiment, the foot-shape appliance was inverted after the substrate was attached to itself so that the seams were on the inside of the appliance. As with two (or more) pieces that may be joined together to form an appliance of the present invention, a single piece may be joined to itself using any of the approaches discussed above (e.g., to form the foot appliance, a sleeve, etc.).

### Representative Formulations or Compositions For Use With an Appliance of the Present Invention

Formulations or compositions that may be used with an appliance of the present invention include emulsifiers, surfactants, viscosity modifiers, natural moisturizing factors, antimicrobial actives, pH modifiers, enzyme inhibitors/inactivators, suspending agents, pigments, dyes, colorants, buffers, perfumes, antibacterial actives, antifungal actives, pharmaceutical actives, film formers, deodorants, opacifiers, astringents, solvents, organic acids, preservatives, drugs, vitamins, aloe vera, and the like.

In some versions of the invention, a clinically beneficial additive of the formulation or composition may either interact directly with epithelial tissue at the cellular level to provide a benefit to the skin, or alternatively, may interact with components at or near the skin surface in order to provide a benefit to the skin.

In one embodiment, the clinically beneficial additive may be an emollient, which is herein defined as an agent that helps restore dry skin to a more normal moisture balance. Emollients act on the skin by supplying fats and oils that blend in with skin, making it pliable, repairing some of the cracks and fissures in the stratum corneum, and forming a protective film that traps water in the skin (i.e., acts as an occlusive agent to occlude water). Emollients that may be suitable for use with the present invention include beeswax, butyl stearate, cermides, cetyl palmitate, eucerit, isohexadecane, isopropyl palmitate, isopropyl myristate, mink oil, mineral oil, nut oil, oleyl alcohol, petroleum jelly or petrolatum, glyceral stearate, avocado oil, jojoba oil, lanolin (or woolwax), lanolin derivatives such as lanolin alcohol, retinyl palmitate (a vitamin A derivative), cetearyl alcohol, squalane, squalene, stearic acid, stearyl alcohol, myristal myristate, certain hydrogel emollients, various lipids, decyl oleate and castor oil.

A preferred clinically beneficial additive is a humectant, which is herein defined to be an agent that supplies the skin with water by attracting moisture from the air and holding it on the skin. Humectants that may be suitable for use with the present invention include alanine, glycerin, PEG, propylene glycol, butylenes glycol, glycerin (glycol), hyaluronic acid, Natural Moisturizing Factor (a mixture of amino acids and salts that are among the skin's natural humectants), saccharide isomerate, sodium lactate, sorbitol, urea, and sodium PCA.

Other clinically beneficial agents that may be suitable for use with the present invention include antioxidants, a unique group of substances that protect a body or other objects from oxidizing. Antioxidants prevent or slow the oxidation process, thereby protecting the skin from premature aging. Exemplary antioxidants for use in the present invention include ascorbic acid ester, vitamin C (ascorbic acid), vitamin E (lecithin), Alpha-Glycosyl Rutin (AGR, or Alpha Flavon, a plant-derived antioxidant), and coenzyme Q10 (also known as ubiquinone).

Other clinically beneficial agents which may be delivered to the skin during use include chelating agents, such as EDTA; absorptive/neutralizing agents, such as kaolin, hectorite, smectite, or bentonite; other vitamins and vitamin sources and derivatives, such as panthenol, retinyl palmitate, tocopherol, and tocopherol acetate; and anti-irritants such as chitin and chitosan.

Additional examples of beneficial agents include skin conditioners, which are herein defined as agents that may help the skin retain moisture, improve softness, or improve texture. Skin conditioners include, for example, amino acids, including alanine, serine, and glycine; allantoin, keratin, and methyl glucose dioleate; alpha-hydroxy acids, including lactic acid and glycolic acid, which act by loosening dead skin cells from the skin's surface; moisturizers (agents that add or hold water in dry skin), including echinacea (an extract of the coneflower plant), shea butter, and certain silicones, including cyclomethicon, dimethicone, and simethicone.

Other examples of beneficial botanical agents, extracts, or other materials that may be suitable for use with the present invention include almonds, chamomile extracts such as bisabolol (believed to relieve irritation, swelling and itching in the skin), elder flowers, honey, safflower oil, and elastin (safflower oil and elastin are believed to aid in retaining skin elasticity).

In addition to one or more clinically beneficial additives, other additives may be included in the formulation or composition. For example, a silicone polymer may be included to improve the slip characteristics of the elastomeric article. Possible silicone polymers include reactive silicones, non-reactive silicones, or a mixture of reactive and non-reactive silicones. Suitable silicones may include, for example, aminosilicones, polyether-modified amino silicones, amino- substituted siloxanes having terminal hydroxy groups, epoxy silicones, quaternary silicones, dimethicone, silicone polyethers, polyether epoxy silicones, silanol fluids, polysildxy linoleyl pyrrolidone phospholipids, and combinations of possible silicones.

Other additives may be included, for example, glucose derived polymers, or mixtures containing glucose derived polymers (e.g., lauryl glucoside available from Cospha under the trade designation Planteran PS 400), silica, silica dispersions, wetting agents, and preservatives (i.e., parabens, such as methylparaben and propylparaben). In one embodiment, the personal-care composition may include emulsion stabilizers. Exemplary emulsion stabilizers include aluminum stearate, magnesium sulfate, hydrated silica, and ozokerite.

In another embodiment a beneficial agent may be held in the formulation or composition in liposomes. A liposome is a vehicle for delivering agents to the skin. More specifically, a liposome is a microscopic sphere formed from a fatty compound, a lipid, surrounding a water-based agent, such as a moisturizer or an emollient. When the liposome is rubbed into the skin, it releases the agent throughout the stratum corneum.

In another embodiment, the beneficial agent may be present in the carrier in the form of a microencapsulant. A microencapsulant is a sphere of an emollient surrounded by a gelatin membrane that prevents the emollient from reacting with other ingredients in the coating composition and helps distribute the emollient more evenly when pressure is applied and the membrane is broken. The process of forming these beads is called microencapsulation and is generally known in the art.

The formulation or composition of the present invention may be applied to the appliance as an aqueous solution, a dispersion, or an emulsion. In one embodiment, an aqueous composition may be formed including from about 4.5% to about 6% by weight of a humectant. In other embodiments, the humectant may be present at 30% or more by weight. In some other embodiments, the humectant may be present at about 10 to about 20% by weight. In still other embodiments, the humectant is present at about 5 to about 40% by weight. This composition may then be applied to the interior surface of an appliance of the present invention.

In one embodiment, the personal-care composition may be applied as an emulsion. In one embodiment, the formulation or composition may be applied to the surface of the appliance as a micro-emulsion. A micro-emulsion is a particularly fine-particle emulsion that can be applied in a spray form. The particle size of a micro-emulsion is generally less than about one micron, whereas traditional emulsions demonstrate particle sizes of greater than about 50 microns.

The components of a formulation or composition may be applied or associated n combination or separately to the surface of the appliance. For example, a 100% humectant composition may be applied, followed by another 100% beneficial additive composition, such that the two (or more) separate applications together form the coating of the appliance. In such a manner, layers of additives may be built up on the surface of the appliance.

The coating may be deposited on the interior surface of the appliances by any suitable method. For example, the appliances may be dipped in the coating. In an alternative embodiment, the appliances may be tumbled in the coating. In various embodiments, the coating may be applied to the surface of the appliance through dipping, immersion, spraying, patting, printing, or any other application method known in the art.

In one embodiment, the coating may be sprayed onto a skin-contacting surface of the appliance. For instance, appliances may be placed in a tumbling apparatus while a solution of the coating is sprayed on the gloves. In one embodiment, the spraying process may be repeated. For instance, the spraying process may be repeated up to about twenty times to coat the inner surface of the gloves. In one embodiment, the spraying process may be carried out for a total of between about ten and about twenty times.

### Representative Marketing and/or Packaging of Appliances of Present Invention

The manufacturer of an appliance of the present invention may fashion messages, statements, or copy to be transmitted to a purchaser, consumer, or user of said appliance. Such messages, statements, or copy may be fashioned to help facilitate or establish an association in the mind of a user of the appliance between an appliance of the present invention, or use thereof, and one or more mental states, psychological states, or states of well being. The communication, statements, or copy may include various alphanumeric strings, including, for example: “moisture”, “moisturize”, “moisturizing”, “pamper”, “pampering”, “ritual”, “personal”, “spa”, “treatment”, “foot”, “hand”, “system”, “effective”, “convenient”, “disposable”, “botanical”, “vitamin”, “relax”, “peace”, “energy”, “energize”, “sex”, “sensuality”, “sensual”, “spirit”, “spiritual”, “clean”, “fresh”, “mountain”, “country”, “zest”, “sea”, “sky”, “health”, “hygiene”, “water”, “waterfall”, or derivatives or combinations thereof. It should be noted that each term appearing in quotes in the preceding list may be in any font, style, color, etc.—and the quotes likely would not appear around the term when the term is employed. These alphanumeric strings may be used either alone, adjacent to, or in combination with, other alphanumeric strings. In one embodiment, the communication, statements, or copy associate an appliance of the present invention and a registered or common-law trademark, name, brand name, and/or logo of the seller or manufacturer of the appliance (and/or health-and-beauty products generally).

The communication, statements, message, or copy could take the form of (i.e., be embodied in a medium such as) a newspaper advertisement, a television advertisement, a radio or other audio advertisement, items mailed directly to addressees, items emailed to addresses, Internet Web pages or other such postings, free standing inserts, coupons, various promotions (e.g., trade promotions), co-promotions with other companies, copy and the like, boxes and packages containing the product (in this case an appliance of the present invention), and other such forms of disseminating information to consumers or potential consumers. Other exemplary versions of such communications, statements, messages, and/or copy may be found in, for example, U.S. Pat. Nos. 6,612,846 and 6,896,521, both entitled “Method for Displaying Toilet Training Materials and Display Kiosk Using Same”; co-pending U.S. application Ser. No. 10/831,476, entitled “Method of Enunciating a Pre-Recorded Message Related to Toilet Training in Response to a Contact”; co-pending U.S. Application Serial Number 10/956763, entitled “Method of Manufacturing and Method of Marketing Gender-Specific Absorbent Articles Having Liquid-Handling Properties Tailored to Each Gender”; each of which is incorporated by reference in their entirety in a manner consistent herewith.

It should be noted that when associating statements, copy, messages, or other communications with a package (e.g., by printing text, images, symbols, graphics, color(s), or the like on the package; or by placing printed instructions in the package; or by associating or attaching such instructions, a coupon, or other materials to the package; or the like) containing appliances of the present invention, the materials of construction of said package may be selected to reduce, impede, or eliminate the passage of water or water vapor through at least a portion of the package. As noted above, a preferred composition for an appliance of the present invention comprises water and a humectant. Therefore packages, containers, envelopes, bags, and the like that reduce, minimize, or eliminate the evaporation or transmission of water or water vapor from appliances contained therein is beneficial. Furthermore, appliances may be individually wrapped in containers, packets, envelopes, bags, or the like that inhibit, reduce, or eliminate the passage or transmission of water or water vapor from appliances contained therein. For purposes of this application, “packages,” “containers,” “envelopes,” “bags,” “packets,” and the like are interchangeable in the sense that they refer to any material adapted to enclose and hold either individual appliances (as in, for example, an individual packet containing a single appliance), or a plurality of appliances (as in a flexible bag made of film containing a plurality of appliances, whether or not each of the individual appliances are enclosed and held in a separate material—such as individual packets).

These and other modifications and variations to the present invention may be practiced by those of ordinary skill in the art, without departing from the spirit and scope of the present invention, which is more particularly set forth in the appended claims. Furthermore, those of ordinary skill in the art will appreciate that the foregoing description is by way of example only, and is not intended to limit the invention so further described in such appended claims.

### EXAMPLES

### Example 1

### Representative Example of a Personal-Care Composition

An exemplary personal-care composition was prepared having the ingredients/components and proportions identified below:

| Component | Weight % | Supplier | Address |
|---|---|---|---|
| Water | 66.1 | N/A | N/A |
| Emulgade CM | 20.0 | Cognis | 300 Brookside Ave, |
| | | | Ambler, PA 19002 |
| Glycerin (99.7% | 4.0 | Glenn Corp. | 4886 Highway 61 N, |
| USP) | | | St. Paul, MN 55110 |
| Hispagel 200 | 4.8 | Cognis | 300 Brookside Ave, |
| | | | Ambler, PA 19002 |
| Sepigel 501 | 3.2 | Seppic | 30, Two Bridges |
| | | | Road, Fairfield, New |
| | | | Jersey 07004 |
| Mackernium-007 | 1.0 | McIntyre Group | 24601 Governors |
| | | | Highway, University |
| | | | Park, IL 60466 |
| Paragon III | 0.5 | McIntyre Group | 24601 Governors |
| | | | Highway, University |
| | | | Park, IL 60466 |
| Fragrance | 0.05 | | |
| Tween 40 | 0.05 | Uniquema | 76 East 24^{th} St, |
| | | | Paterson, NJ 07544 |
| Sodium citrate | 0.3 | Sigma-Aldrich | 3050 Spruce Street, |
| 20% | | | St. Louis, MO 63103 |

The recited proportions of water, glycerin, Emulgade CM, Mackernium-007, and Paragon III were mixed together a Lightnin Labmaster mixer LIU10F (135 Mt. Read Blvd., Rochester, N.Y.). Tween 40 and the fragrance were mixed separately in a small container using a spatula. The fragrance/Tween 40 mixture was then mixed into the mixture containing water, glycerin, and the other ingredients identified above. Hispagel 200 and Sepigel 501 were then added in sequence to the resulting combination in an Ultra-Turrax T50 Basic high-shear homogenizer (IKA® Works, 2635 Northchase Pkwy. SE, Wilmington, N.C. 28405). Finally, pH of the formulation was adjusted by adding sodium citrate until a pH of 6.0 was achieved, as measured using a SevenMulti pH meter (Mettler-Toledo, 1900 Polaris Parkway, Columbus, Ohio, 43240).

### Example 2

### Representative Example of a Personal-Care Composition

An exemplary personal-care composition having the ingredients and proportions identified below was prepared:

| Component | Weight % | Supplier | Address |
|---|---|---|---|
| Water | 67.1 | N/A | N/A |
| Emulgade CM | 20.0 | Cogins | 300 Brookside Ave, |
| | | | Ambler, PA 19002 |
| Glycerin (99.7% | 4.0 | Glenn Corp. | 4886 Highway 61 N, |
| USP) | | | St. Paul, MN 55110 |
| Pentavitin | 4.0 | Pentapharm/ | 20 Glover Ave, |
| (saccharide | | CenterChem | Norwalk, CT 06850 |
| isomerate) | | | |
| Sepigel 501 | 2.5 | Seppic | 30, Two Bridges |
| | | | Road, Fairfield, New |
| | | | Jersey 07004 |
| Panthenol | 1.0 | Sigma-Aldrich | 3050 Spruce Street, |
| | | | St. Louis, MO 63103 |
| Paragon III | 0.5 | McIntyre Group | 24601 Governors |
| | | | Highway, University |
| | | | Park, IL 60466 |
| Keltrol CG | 0.3 | CPKelco | 1000 Parkwood |
| (Xanthan Gum) | | | Circle, Atlanta, GA |
| | | | 30339 |
| Fragrance | 0.2 | | |
| Tween 40 | 0.2 | Uniquema | 76 East 24^{th} St, |
| | | | Paterson, NJ 07544 |
| Sodium citrate | 0.2 | Sigma-Aldrich | 3050 Spruce Street, |
| 20% | | | St. Louis, MO 63103 |

The recited proportion of xanthan gum was dispersed in water by thoroughly mixing the material in a Lightnin Labmaster mixer LIU10F (135 Mt. Read Blvd., Rochester, N.Y.) at a setting of 400 rpm until the gum was fully hydrated (approximately an hour). Glycerin, Emulgade CM, and Pentavitin, followed by panthenol and Paragon III were then mixed into the xanthan-gum/water mixture. Sepigel 501 was then added to the combination and homogenized using a high shear mixer (Ultra-Turrax T50 Basic, IKA® Works, 2635 Northchase Pkwy. SE, Wilmington, N.C. 28405). Tween 40 and the fragrance were mixed separately in a small container using a spatula. The fragrance/Tween 40 mixture was then mixed into the combination. Finally, pH of the formulation was adjusted by adding sodium citrate until a pH of 6.0 was achieved, as measured using a SevenMulti pH meter (Mettler-Toledo, 1900 Polaris Parkway, Columbus, Ohio, 43240).

### Example 3

### Representative Example of a Personal-Care Composition

An exemplary personal-care composition was prepared having the ingredients and proportions identified below was prepared:

| Component | Weight % | Supplier | Address |
|---|---|---|---|
| Water | 73.0 | N/A | N/A |
| Glycerin (99.7% | 5.0 | Glenn Corp. | 4886 Highway 61 N, |
| USP) | | | St. Paul, MN 55110 |
| Cognis IPP | 5.0 | Cognis | 300 Brookside Ave, |
| | | | Ambler, PA 19002 |
| Arlacel 165 | 3.0 | Uniquema | 76 East 24^{th} St, |
| | | | Paterson, NJ 07544 |
| Petrolatum (Super | 3.0 | Crompton | 771 Old Saw Mill |
| White Protopet) | | | River Road, |
| | | | Tarrytown, NY 10531 |
| Lipex 512 (Shea | 2.0 | Jarchem Industries | 414 Wilson Ave, |
| Butter) | | | Newark, NJ 07105 |
| BioVera Oil (Aloe | 2.0 | BioChemical | 498 Kingston Road, |
| Vera) | | International | Satellite Beach, FL 32937 |
| DC 200 Fluid, 100 cst | 2.0 | Dow Corning | PO Box 994, 2200 |
| | | | West Salzburg Road, |
| | | | Midland, MI 48686 |
| Corpure Avocado | 1.0 | Croda | 7 Century Drive, |
| (Avocado Oil) | | | Parsippany, NJ |
| | | | 07054 |
| Cetyl Alcohol (NF) | 1.0 | Glenn Corp. | 4886 Highway 61 N, |
| | | | St. Paul, MN 55110 |
| Emerest 2400 | 0.5 | Cognis | 300 Brookside Ave, |
| (Glyceryl Stearate) | | | Ambler, PA 19002 |
| dl-alpha Tocopherol | 0.5 | Ruger Chemical | 1515 W. Blancke |
| Acetate (Vitamin E | | | Street, Linden, NJ |
| acetate) | | | 07036 |
| Paragon III | 0.5 | McIntyre Group | 24601 Governors |
| | | | Highway, University |
| | | | Park, IL 60466 |
| Actiphyte of | 0.5 | Active Organics | 1097 Yates Street, |
| Chamomile AQ | | | Lewisville, TX 75057 |
| (Chamomile Extract) | | | |
| Ketrol CG (Xanthan | 0.2 | CPKelco | 1000 Parkwood |
| Gum) | | | Circle, Atlanta, GA |
| | | | 30339 |
| Fragrance | 0.2 | | |
| Tween 40 | 0.2 | Uniquema | 76 East 24^{th} St, |
| | | | Paterson, NJ 07544 |
| Sodium Citrate 20% | 0.2 | Sigma-Aldrich | 3050 Spruce Street, |
| solution | | | St. Louis, MO 63103 |
| Versene NA | 0.1 | Dow Chemical | PO Box 1206, |
| (Disodium EDTA) | | | Midland, MI 48642 |
| BHT | 0.1 | Universal Preserv-A- | 33 Truman Drive |
| | | Chem | South, Edison, NJ 08817 |

The recited proportion of xanthan gum was dispersed in water by thoroughly mixing the material in a Lightnin Labmaster mixer LIU10F (135 Mt. Read Blvd., Rochester, N.Y.) at a setting of 400 rpm until the gum was fully hydrated for approximately one hour. Each of the aqueous ingredients—glycerin, Paragon III, Actiphyte of Chamomile AQ, and Versene NA—were then added to the water phase. (This complete formulation is an oil-in-water (o/w) emulsion where all water-soluble ingredients are mixed separately and called “the water phase”; and the same is done for the oil-soluble ingredients denoted as the “oil phase.”) Tween 40 and the fragrance were mixed separately in a small container using a spatula. The fragrance/Tween 40 mixture was then mixed into the combination. The water phase was then heated to 76 degrees Celsius using a VWR hotplate (1310 Goshen Parkway, West Chester, Pa. 19380). Ingredients for the oil phase of the formulation were then mixed in a separate container using the Lightnin Labmaster and also heated to 76 degrees Celsius on a hotplate. The ingredients were Cognis IPP, Arlacel 165, Petrolatum, Lipex 512, BioVera Oil, DC 200 Fluid, Corpure Avocado, Cetyl Alcohol, Emerest 2400, dl-alpha tocopherol acetate, and BHT. The oil-phase mixture was then added to the water-phase mixture, both at the recited temperature of 76 degrees Celsius. The combination was mixed in a container at a setting of 400 rpm. After mixing at this speed for approximately 10 minutes, the rotational speed was increased to 470 rpm for an additional 5 minutes. Then the combination was homogenized for three minutes using a high-shear mixer (Ultra-Turrax T50 Basic, IKA® Works, 2635 Northchase Pkwy. SE, Wilmington, N.C. 28405). The combination was then allowed to cool down with the Lightnin Labmaster mixer LIU10F (135 Mt. Read Blvd., Rochester, N.Y.) set at a rotational speed of 400 rpm. Finally, pH of the formulation was adjusted by adding sodium citrate until a pH of 6.0 was achieved, as measured using a SevenMulti pH meter (Mettler-Toledo, 1900 Polaris Parkway, Columbus, Ohio, 43240).

### Example 4

### Representative Example of a Personal-Care Composition

An exemplary personal-care composition was prepared having the ingredients and proportions identified below was prepared:

| Component | % | Supplier | Address |
|---|---|---|---|
| Water | 63.7 | N/A | N/A |
| Emulgade CM | 20.0 | Cognis | 300 Brookside Ave, |
| | | | Ambler, PA 19002 |
| Glycerin (99.7% USP) | 4.0 | Glenn Corp. | 4886 Highway 61 N, |
| | | | St. Paul, MN 55110 |
| Hispagel 200 | 4.8 | Cognis | 300 Brookside Ave, |
| | | | Ambler, PA 19002 |
| Sepigel 501 | 3.2 | Seppic | 30, Two Bridges |
| | | | Road, Fairfield, New |
| | | | Jersey 07004 |
| Mackernium-007 | 1.0 | McIntyre Group | 24601 Governors |
| | | | Highway, University |
| | | | Park, IL 60466 |
| Paragon III | 0.5 | McIntyre Group | 24601 Governors |
| | | | Highway, University |
| | | | Park, IL 60466 |
| Panthenol | 0.5 | Sigma-Aldrich | 3050 Spruce Street, |
| | | | St. Louis, MO 63103 |
| Tindoerm A | 0.5 | Ciba Specialty | 4090 Premier Drive, |
| | | Chemicals | High Point, NC |
| | | | 27261 |
| Actiphyte of Aloe | 0.5 | Active Organics | 1097 Yates Street, |
| Vera extract 10 fold | | | Lewisville, TX 75057 |
| Actiphyte of Avocade | 0.25 | Active Organics | 1097 Yates Street, |
| | | | Lewisville, TX 75057 |
| Actiphyte of Jojoba | 0.25 | Active Organics | 1097 Yates Street, |
| Meal | | | Lewisville, TX 75057 |
| Fragrance | 0.2 | | |
| Tween 40 | 0.2 | Uniquema | 76 East 24^{th} St, |
| | | | Paterson, NJ 07544 |
| Tinoderm E | 0.1 | Ciba Specialty | 4090 Premier Drive, |
| | | Chemicals | High Point, NC |
| | | | 27261 |
| Sodium citrate 20% | 0.3 | Sigma-Aldrich | 3050 Spruce Street, |
| | | | St. Louis, MO 63103 |

The recited proportions of water, glycerin, Emulgade CM, Mackernium-007, Tinoderm A, Tinoderm E, Aloe Vera, Avocado, Jojoba Meal and Paragon III were mixed together a Lightnin Labmaster mixer LIU10F (135 Mt. Read Blvd., Rochester, N.Y.). Tween 40 and the fragrance were mixed separately in a small container using a spatula. The fragrance/Tween 40 mixture was then added to the previous combination. Hispagel 200 and Sepigel 501 were then added in sequence to the resulting combination in an Ultra-Turrax T50 Basic high-shear homogenizer (IKA® Works, 2635 Northchase Pkwy. SE, Wilmington, N.C. 28405). Finally, pH of the formulation was adjusted by adding sodium citrate until a pH of 6.0 was achieved, as measured using a SevenMulti pH meter (Mettler-Toledo, 1900 Polaris Parkway, Columbus, Ohio, 43240).

### Example 5

### Prophetic; Representative Appliances

Exemplary appliances are prepared in the following manner.

First, a liquid-impermeable layer is prepared by producing a cast film from Kraton 6638 polymer resin (Kraton 6638 is a blend of 80% by weight Kraton 1730 styrene-(ethylene-propylene)-styrene-(ethylene-propylene) tetrablock copolymer from Kraton Polymers LLC, 7% by weight PETROTHANE NA601 polyethylene wax from Quantum Chemical Co., and 13% by weight REGALREZ 1126 tackifier from Eastman Chemical Co). As the film is being produced (at a 75 grams per square meter basis weight prior to stretching), it is stretched by about 400% along the dimension parallel to the direction of travel of the film. To one side of the film, a necked polypropylene spunbond materials, having a basis weight of 0.8 ounces per square yard, is attached. This is accomplished by first spraying a hot-melt adhesive, #2840 (available from Ato-Findley), at an add-on level of 2 grams per square meter, and heated to a temperature of 365 degrees Fahrenheit, to the side of the spunbond materials that is to contact the film. The film and nonowoven are directed to a nip between to rolls such that the spunbond material is adhesively bonded to the film, thereby creating a 2-layer laminate. The laminate was then allowed to retract, thereby gathering the spunbond material.

Once the substrate is prepared, it is cut into individual pieces in the shape of hand and foot (similar to the shapes representatively depicted in FIGS. 2, 2A, 3, and 3A above). The individual pieces are then sewn together to make either a glove into which a user could insert his or her hand, or a sock into which a user could insert his or her foot.

A personal-care composition is then applied. Each of the formulations identified in Examples 1, 2, 3, and 4 above are added to the socks and gloves. The formulations are applied to the exposed surfaces of the appliances (with the seam visible) using a syringe (4 grams) and a spatula is used to spread the formulation to cover all parts of the product. The product is then inverted back with the seams inside and placed in air-tight bags to prevent evaporation until the time of use.

### Example 6

### Prophetic; Representative Appliances

The prophetic, exemplary appliance of Examples 5 is prepared, with the exception that the film and fibrous layers of Example 5 are thermally bonded to one another without an adhesive. Methods disclosed elsewhere in the application, and known in the art of thermal bonding, are used to join the referenced materials to form the described appliances.

We claim:

1. An appliance adapted to transfer a composition from the interior surface of the appliance to the skin of a wearer of the appliance, the appliance comprising:
a first substrate having a perimeter, the first substrate comprising a first liquid-impermeable layer attached to a first fibrous layer;
a second substrate having a perimeter, the second substrate comprising: a second liquid-impermeable layer attached to a second fibrous layer;
wherein the first substrate and the second substrate are attached to one another in a way that defines an interior volume into which a wearer of the appliance may insert a portion of his or her body, said interior volume bounded in part by an interior surface; and
a composition associated with at least a portion of the interior surface, wherein the first liquid-impermeable layer is interposed between the composition and the first fibrous layer, and the second liquid-impermeable layer is interposed between the composition and the second fibrous layer.

2. The appliance of claim 1 wherein the perimeter of the first substrate, the perimeter of the second substrate, or the perimeters of the first and second substrates define a shape of a human hand.

3. The appliance of claim 1 wherein the composition is adapted to moisturize the skin of a user of the appliance.

4. The appliance of claims 3 wherein the composition comprises water and a humectant.

5. The appliance of claim 3 wherein the composition comprises an ingredient adapted to occlude liquid.

6. The appliance of claim 1 wherein the first fibrous layer is attached to the first liquid-impermeable layer at discrete locations, and wherein the second fibrous layer is attached to the second liquid-impermeable layer at discrete locations.

7. The appliance of claim 6 wherein the first fibrous layer and the second fibrous layer are gathered.

8. The appliance of claim 1 wherein the first liquid-impermeable layer and second liquid-impermeable layer are elastomeric films.

9. The appliance of claim 1 wherein the appliance is adapted to be disposed of after a single use.

10. A package, the package comprising:
a plurality of appliances of claim 1, each contained in a liquid-impermeable envelope; and
a container for the plurality of appliance-containing envelopes.

11. The package of claim 10 further comprising a second personal-care article of manufacture.

12. The package of claim 11 further comprising a statement disposed in, on, or proximate to said container, wherein the statement associates the appliance with one or more of the following: “moisture”, “moisturize”, “moisturizing”, “pamper”, “pampering”, “ritual”, “personal”, “spa”, “treatment”, “foot”, “hand”, “system”, “effective”, “convenient”, “disposable”, “botanical”, “vitamin”, “relax”, “peace”, “energy”, “energize”, “sex”, “sensuality”, “sensual”, “spirit”, “spiritual”, “clean”, “fresh”, “mountain”, “country”, “zest”, “sea”, “sky”, “health”, “hygiene”, “water”, “waterfall”.

13. An appliance adapted to transfer a composition from the appliance to the skin of a wearer of the appliance, the appliance comprising:
a substrate having a perimeter, the substrate comprising a liquid-impermeable layer attached to a fibrous layer, wherein the substrate is attached to itself in a way that defines an interior volume into which a wearer of the appliance may insert a portion of his or her body, said interior volume bounded in part by an interior surface;
a composition associated with at least a portion of the interior surface, wherein the liquid-impermeable layer is interposed between the composition and the fibrous layer.

14. The appliance of claim 13 wherein the appliance is adapted to receive a foot of a user of the appliance.

15. The appliance of claim 13 wherein the composition is adapted to moisturize the skin of a user of the appliance.

16. The appliance of claim 15 wherein the composition comprises water and a humectant.

17. The appliance of claim 13 wherein the fibrous layer is a nonwoven.

18. The appliance of claim 13 wherein the fibrous layer is attached to the liquid-impermeable layer at discrete locations.

19. The appliance of claim 18 wherein the fibrous layer is gathered.

20. A package, the package comprising:
a plurality of appliances of claim 13, each contained in a liquid-impermeable envelope; and
a container for the plurality of appliance-containing envelopes.

21. A message adapted to be communicated to consumers, wherein the message is based, in whole or in part, on information relating to an appliance of claim 1 or 13, and wherein the message is contained in text, a symbol, a graphic, an image, and/or color, and wherein the message is embodied in a medium capable of being transmitted to consumers.

22. An appliance adapted to transfer a composition from the appliance to the skin of a wearer of the appliance, the appliance comprising:
a substrate having a perimeter, the substrate comprising a liquid-impermeable layer attached to a fibrous layer; and
a composition associated with the liquid-impermeable layer, wherein the liquid-impermeable layer is interposed between the composition and the fibrous layer.

23. The appliance of claim 22 wherein the appliance is a patch adapted to releasably engage the skin of a user of the appliance.

24. The appliance of claim 23 wherein the appliance comprises an adhesive adapted to releasably engage the skin of a user of the appliance.
